(19) United States
(12) Patent Application Publication
(10) Pub. No.: US 2005/0054840 A1
(43) Pub. Date: Mar. 10, 2005

(54) Method for the production of azo compounds

(76) Inventor: Jean-Michel Bossoutrot, Chaponost (FR)

Correspondence Address: Steven D Boyd;Atofina Chemicals Inc, 2000 Market Street, 26th Floor, Philadelphia, PA, 19103, US

(21) Appl. No.: 10/482,150

(22) PCT Filed: Apr. 29, 2002
(86) PCT No.: PCT/FR02/01488

(30) Foreign Application Priority Data
Jun. 28, 2001 (FR) 01/08541

(51) Int. Cl. C07C 253/30

(52) U.S. Cl. 534/586

(57) ABSTRACT

The invention relates to a method for production of azo compounds. In particular the invention relates to a step-wise method for production of an azo compound, by seeding and oxidation of the corresponding hydrazoic compound.

### FIELD OF THE INVENTION

The present invention relates to a method of preparation of azo compounds.

### BACKGROUND OF THE INVENTION

Azo compounds, and notably 2,2′-azobis(isobutyronitrile), are well-known products that are used notably as a blowing agent or synthesis intermediate or initiator of polymerization reactions employing free radicals.

These reactions can be reactions of bulk polymerization, solution polymerization, suspension polymerization or emulsion polymerization, and make use of a great variety of monomers, for example (meth)acrylic monomers, vinylic monomers such as acrylamide, acrylonitrile, alkyl (meth)acrylate, styrene, vinyl acetate and chloride, vinylidene chloride. The fields of application are therefore very varied and relate notably (but not exclusively) to acrylic sheets or fibers, flocculents, paints, coating resins, grafted polyols, polystyrene, PVC, PVA, PMMA.

Azo compounds are generally obtained by oxidation of the corresponding hydrazo derivatives. After oxidation, the resulting suspension is drained, then dried to give a solid in the form of powder with average grain size generally between 20 and 110 μm. For example, 2,2′-azobis(isobutyronitrile) obtained by reacting the corresponding hydrazo derivative with chlorine, in an aqueous medium, is in the form of powder with average grain size of about 45 μm after drying and the grain size at 10 wt. % (d10) is about 20 μm.

Now, azo compounds, in the form described above, pose many problems:
they generate dust that may present a risk of explosion and/or an industrial health risk,
they have poor castability
and problems of lumping are often encountered during storage.

The problems identified above are solved partly or completely by the invention. The invention supplies a global solution that makes it possible to increase the average grain size of an azo compound relative to that of a compound obtained by a conventional method of manufacture. The average grain size can be doubled or even trebled.

### SUMMARY OF THE INVENTION

The invention therefore provides a method of manufacture of an azo compound (A), starting from the corresponding hydrazo compound (HA), by seeding.

The method according to the invention includes a step in which an oxidizing agent is reacted with a hydrazo compound (HA), in a liquid medium, in the presence of a sufficient quantity of crystals of the corresponding azo compound (seeds).

### DETAILED DESCRIPTION OF THE INVENTION

According to one embodiment, the hydrazo compound is in suspension in the liquid medium.

According to one embodiment, the hydrazo compound is in emulsion in the liquid medium.

This oxidation step can be represented schematically by the following equation: 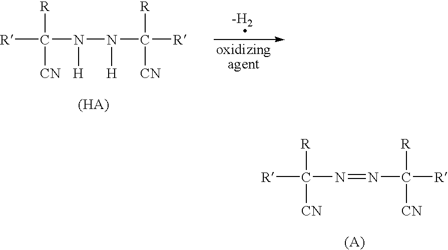
where R and R′ in (HA) and (A) can be identical or different, and they each represent:
a linear or branched alkyl group, preferably a C₁-C₆ alkyl group, possibly substituted by a hydroxy, alkoxy or carboxy group or by a halogen atom, or
a cycloalkyl group preferably with 3 to 6 carbon atoms, possibly substituted by a hydroxy, alkoxy or carboxy group or by a halogen atom, or
an aryl group such as phenyl or naphthyl, possibly substituted by a hydroxy, alkyl, alkoxy or carboxy group or by a halogen atom, or
an aralkyl group such as benzyl or phenethyl, possibly substituted by one or more alkyl, alkoxy, hydroxy or carboxy groups, or by one or more halogen atoms; or alternatively R and R′ form, with the carbon atom to which it is (or they are) joined, a cycloalkyl radical.

As examples of compound (A) we may mention 2,2′-azobis isobutyronitrile (R═R′═CH₃), 2,2′-azobis(2,4-dimethyl-valeronitrile), 2,2′-azobis(2-methylbutyronitrile), 1,1′-azobis(1-cyclohexanecarbonitrile) and 4,4′-azobis(4-cyanopentanoic acid).

As examples of oxidizing agent we may mention chlorine, oxygen, hydrogen peroxide and ozone.

Preferably, the liquid medium is aqueous (i.e. consisting essentially of water).

In addition to the reactants (HA and oxidizing agent), the liquid reaction medium can contain a catalyst, for example bromine ions. It can also contain additives, such as surfactants, for example sodium bis sulfosuccinate, notably sodium bis(2-ethylhexyl)-sulfosuccinate.

The temperature of the reaction medium is generally close to room temperature. It is preferably between 15 and 25° C. and advantageously between 18 and 20° C.

The seeds or crystals of compound (A) can be present in the reaction medium before the start of the oxidation reaction and/or can be introduced during the oxidation reaction.

After the oxidation step, the resulting suspension is drained and then the solid obtained is washed and finally dried.

According to a preferred embodiment of the invention, dihydrocitogen (R═R′═CH₃), in suspension in an aqueous medium, is oxidized with chlorine in the presence of a sufficient quantity of crystals of 2,2′-azobis(isobutyronitrile) (AIBN) with average grain size advantageously between 110 and 180 μm and better still between 110 and 150 μm.

Preferably, these crystals of AIBN (seeds) are present at the start of the oxidation step and represent between 0.5 and 20 wt. %, advantageously between 5 and 15 wt. % relative to the reaction medium (i.e. water+reactants+additives+seeds).

The dihydrocitogen suspended in the aqueous medium represents preferably between 2 and 30 wt. %, and advantageously between 5-15 wt. % relative to the reaction medium (i.e. water+reactants+additives+seeds).

The reaction medium can be stirred by any known means.

The temperature of the reaction medium is preferably between 15 and 25° C. and advantageously between 18 and 20° C.

The duration of the oxidation step according to the invention varies depending on the desired granulometry. It is preferably between 2 and 6 hours.

Preferably, the chlorine is injected into the aqueous medium continuously, in gaseous form, throughout the oxidation.

At the end of the oxidation step according to the invention, the product obtained is drained, then washed and finally dried to give AIBN of the desired average grain size.

The AIBN crystals (seeds) can be prepared starting from dihydrocitogen by a conventional method of oxidation, followed by a step (r1) in which dihydrocitogen is reacted with chlorine in an aqueous medium containing a proportion of the suspension resulting from the conventional method (conventional method of oxidation).

Depending on the average grain size of AIBN desired, dihydrocitogen can be reacted again (r2) with chlorine in an aqueous medium containing a proportion of the suspension resulting from the preceding step (r1).

Advantageously, the seeds can be obtained by reacting n times, n being an integer greater than 2, preferably in the range from 3 to 5, dihydrocitogen with chlorine in an aqueous medium containing each time a proportion of the suspension resulting from the preceding oxidation reaction.

Preferably, the aqueous medium for the reaction step (rn) contains half the quantity of the suspension resulting from the reaction step (r(n-1)).

The preparation of seeds of AIBN described above can be applied to other azo compounds A, oxidizing agents and liquid medium.

When the average grain size of the seeds intended for the oxidation step according to the invention is reached, it is not necessary to proceed as described above for making new seeds. It is sufficient, after each oxidation batch, to discharge just a proportion of the resulting suspension and leave the rest of it for the next oxidation step. This makes it possible to deliver seeds of intermediate grain size.

It is also possible to use, as seeds, the crystals of compound A obtained after draining a suspension resulting from the oxidation step according to the invention, followed if necessary by drying.

The applicant found that compound A obtained after the oxidation step described above facilitates the subsequent operations of draining and drying and thus makes it possible to increase the productivity of the manufacturing plant. The granulometry of the dried compound A is less spread out and the dried compound A contains less dust and offers better castability.

### EXAMPLES

### Preparation of the Seeds

Test 1

Introduce the following, in succession, into a 1.5-liter reactor equipped with a stirring system that can be used for mixing a suspension:
216 g (1.3 mol) of dry dihydrocitogen
1350 g of water
0.1 g of di(2-ethylhexyl)sulfosuccinate (DOS)
0.6 g of sodium bromide.

Then introduce continuously, by means of a gas feed pipe submerged in the reactor, into the aqueous medium thus formed, 95 g of chlorine over a total time of 5.5 h. During the reaction, keep the medium at a temperature between 18 and 20° C. Stop the stirrer one hour after the end of introduction of the chlorine.

Then filter the suspension, and wash the solid obtained until the pH of the wash water is close to 7.

Finally dry the washed solid under vacuum at 30° C. for 12 h to attain a final moisture content below 0.05 wt. %. The dried solid (crystals of azobis isobutyronitrile (AIBN)) has an average grain size (d50) of 45 μm and a granulometry (d10) of 20 μm. The castability of this solid is very poor.

Test 2

Follow the procedure described in test 1, but adding 25 g of dried solid resulting from test 1 before introducing the chlorine.

After drying, crystals are obtained with average grain size (d50) of 75 μm and with a d10 of 40 μm.

Test 2a

Follow the procedure described in test 2, but using 216 g (instead of 25) of AIBN crystals resulting from test 1.

After drying, crystals are obtained with average grain size (d50) of 60 μm and with a d10 of 35 μm.

Test 3

Follow the procedure described in test 2, but adding 25 g of dried solid resulting from test 2 instead of the solid resulting from test 1.

After drying, crystals are obtained with average grain size (d50) of 85 μm.

Test 4

Follow the procedure described in test 1 and at the end of the reaction only discharge half of the suspension obtained. Then introduce successively into the reactor containing the other half:
216 g (1.3 mol) of dry dihydrocitogen
675 g water
0.1 g of di(2-ethylhexyl)sulfosuccinate (DOS)
0.3 g of sodium bromide.

Then introduce continuously, by means of a gas feed pipe submerged in the reactor, into the aqueous medium thus formed, 95 g of chlorine over a total time of 5.5 h. During the reaction, keep the medium at a temperature between 18 and 20° C. Stop the stirrer one hour after the end of introduction of the chlorine, then again discharge only half of the suspension obtained.

Then introduce the various constituents of the charge indicated above successively into the reactor containing the other remaining half. After stopping the reaction, discharge only half of the suspension obtained and introduce, into the reactor containing the other half, the same charge as for the preceding batch.

After two additional semi-discharges (as indicated above), use half of the final suspension for carrying out example 1 and drain the other half, then wash the solid until the pH of the wash water is close to 7, and finally wash the dried solid to give crystals with average grain size (d50) of 150 μm and with granulometry (d10) of 60 μm.

### Example 1

Introduce successively, into the reactor containing half of the final suspension resulting from test 4:
216 g (1.3 mol) of dry dihydrocitogen
675 g of water
0.1 g of di(2-ethylhexyl)sulfosuccinate (DOS)
0.3 g of sodium bromide.

Then introduce continuously, by means of a gas feed pipe submerged in the reactor, into the aqueous medium thus formed, 95 g of chlorine over a total time of 5.5 h. During the reaction, keep the medium at a temperature between 18 and 20° C. Stop the stirrer one hour after the end of introduction of the chlorine, and filter the suspension. Then filter the solid obtained, and wash it with water until the pH of the wash water is close to 7. Relative to test 1, the filtration time is reduced by 50%.

The residual moisture content of the washed solid is 30% lower relative to that of the solid obtained according to test 1 and the apparent density is increased by 30%.

Finally, dry the washed solid under vacuum at 30° C. for 6 h, to reach a final moisture content below 0.05 wt. %. The dried solid (crystals of azobis isobutyronitrile (AIBN)) has an average grain size (d50) of 150 μm and a granulometry (d10) of 60 μm.

### Example 2

Follow the procedure described in example 1 except that the duration of introduction of the chlorine is 4 h. A product similar to that of example 1 is obtained.

### Example 3

Follow the procedure described in example 1 except that the stirring speed is increased by 50%. A product similar to that of example 1 is obtained.

### Example 4

The operating conditions of example 1 were reproduced on an industrial scale in a 5 m³ reactor. A product similar to that of example 1 was obtained.

### Example C1

Follow the procedure described in test 1 except that the chlorine is introduced over a period of 4 h (instead of 5.5). The dried solid (crystals of azobis isobutyronitrile (AIBN)) has an average grain size (d50) less than that of test 1.

### Example C2

Follow the procedure described in test 1 except that the stirring speed is increased by 50%. An average grain size less than that of the crystals of test 1 is obtained.

### Example 5

Follow the procedure described in test 1 except that, before the start of chlorination, 216 g of crystals of AIBN with average grain size of 150 μm, obtained in example 1, are introduced into the reactor.

After drying, AIBN crystals are obtained with an average grain size (d50) of 150 μm.

1. Method of manufacture of an azo compound (A) comprising a step in which an oxidizing agent is reacted (in accordance with the equation given below) with a hydrazo compound (HA), in a liquid medium, in the presence of a sufficient quantity of crystals of the corresponding azo compound (seeds): 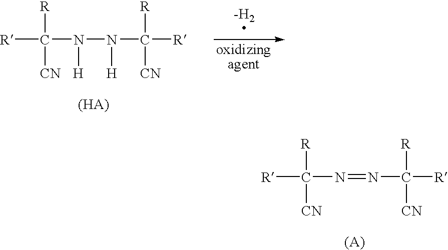
where r and r′ in (ha) and (a) can be identical or different, with each representing:
a linear or branched alkyl group, preferably a C₁-C₆ alkyl group, possibly substituted by a hydroxy, alkoxy or carboxy group or by a halogen atom, or
a cycloalkyl group preferably with 3 to 6 carbon atoms, possibly substituted by a hydroxy, alkoxy or carboxy group or by a halogen atom, or
an aryl group such as phenyl or naphthyl, possibly substituted by a hydroxy, alkyl, alkoxy or carboxy group or by a halogen atom, or
an aralkyl group such as benzyl or phenethyl, possibly substituted by one or more alkyl, alkoxy, hydroxy or carboxy groups, or by one or more halogen atoms; or alternatively R and R′ form, with the carbon atom to which it is (or they are) joined, a cycloalkyl radical.

2. Method as claimed in claim 1, characterized in that compound A comprises 2,2′ azobis isobutyronitrile (R═R′═CH₃), 2,2′-azobis(2,4-dimethyl-valeronitrile), 2,2′-azobis(2-methylbutyronitrile), 1,1′-azobis(1-cyclohexanecarbonitrile) or 4,4′-azobis(4-cyanopentanoic acid).

3. Method as claimed in claim 1, characterized in that the oxidizing agent comprises chlorine, oxygen, hydrogen peroxide or ozone.

4. Method as claimed in claim 1, characterized in that the liquid medium is aqueous.

5. Method as claimed in claim 1, characterized in that the temperature of the reaction medium is between 15 and 25° C.

6. Method as claimed in claim 5, characterized in that the temperature of the reaction medium is between 18 and 20° C.

7. Method as claimed in claim 1, characterized in that after the oxidation step, the resulting suspension is drained then washed and dried.

8. Method as claimed in claim 1, characterized in that the crystals of compound (A) can be prepared starting from the corresponding compound HA by a conventional method of oxidation followed by n reactions, n being an integer greater than 2, in the course of which compound HA is oxidized in a liquid medium containing a proportion of the suspension resulting from the preceding reaction.

9. Method of claim 8 wherein n is an integer greater in the range from 3 to 5.

10. Method of manufacture of 2,2′-azobis(isobutyronitrile) (AIBN) starting from dihydrocitogen, characterized in that dihydrocitogen, in suspension in an aqueous medium, is oxidized with chlorine in the presence of a sufficient quantity of crystals of 2,2′-azobis(isobutyronitrile) with average grain size between 110 and 180 μm.

11. Method as claimed in claim 10 wherein said average grain size advantageously between 110 and 150 μm.

12. Method as claimed in claim 10, characterized in that the crystals of AIBN (seeds) are present at the start of the oxidation step and represent between 0.5 and 20 wt. %, relative to the reaction medium.

13. Method as claimed in claim 12, wherein the crystals of AIBN represent between 5 and 15 wt. %, relative to the reaction medium.

14. Method as claimed in claim 10, characterized in that the dihydrocitogen suspended in the aqueous medium represents between 2 and 30 wt. % relative to the reaction medium.

15. Method as claimed in claim 14, characterized in that the dihydrocitogen suspended in the aqueous medium represents between 5 and 15 wt. % relative to the reaction medium.

16. Method as claimed in claim 10, characterized in that the temperature of the reaction medium is between 15 and 25° C.

17. Method as claimed in claim 16, characterized in that the temperature of the reaction medium is between 18 and 20° C.

18. Method as claimed in claim 10, characterized in that after the oxidation step, the resulting suspension is drained then washed and dried.

19. Method as claimed in claim 10, characterized in that the crystals of compound (A) can be prepared starting from the corresponding compound HA by a conventional method of oxidation followed by n reactions, n being an integer greater than 2, in the course of which compound HA is oxidized in a liquid medium containing a proportion of the suspension resulting from the preceding reaction.

20. Method of claim 19 wherein n is an integer greater in the range from 3 to 5.
